SEQUENCE LISTING (28 sequences)

SEQ ID NO: 1 - DNA, 17 bp, Homo sapiens
acctcaggaaataaacg

SEQ ID NO: 2 - DNA, 17 bp, Homo sapiens
aatatggatgacactgg

SEQ ID NO: 3 - DNA, 17 bp, Homo sapiens
gaatgtcctttgcacgc

SEQ ID NO: 4 - DNA, 17 bp, Homo sapiens
tgtggtgctgctgcccg

SEQ ID NO: 5 - DNA, 17 bp, Homo sapiens
tccctggcatctgttca

SEQ ID NO: 6 - DNA, 17 bp, Homo sapiens
cttataataagctgggg

SEQ ID NO: 7 - DNA, 17 bp, Homo sapiens
agtcgggagcttcgatc

SEQ ID NO: 8 - DNA, 17 bp, Homo sapiens
cttttctgaagagccgg

SEQ ID NO: 9 - DNA, 17 bp, Homo sapiens
cacacagttttgataaa

SEQ ID NO: 10 - DNA, 17 bp, Homo sapiens
gctctgtgaatttgagg

SEQ ID NO: 11 - DNA, 17 bp, Homo sapiens
gctggagcgcccacggt

SEQ ID NO: 12 - DNA, 17 bp, Homo sapiens
ggaagtgccactgggat

SEQ ID NO: 13 - DNA, 2384 bp, Homo sapiens
atctgccaccgcagtctggttggagctgttgtcttgtatgctcagcgaggcccggagagacccgggagagagctaggccgagtccaccgcccgagtctgctgcccgagcccgcgttacgcacaaagccgccgatccccggcctggggtgagcagagcgaccaccgcccgggagcagcgcggcgagacgcacggtgcgccctatgcccccgcgcccccaccgcccccgccgcggcagccgaagcgcagcgagagaacgcgccaccgcggggcccgggtgcagctagcgaccctctcgccacctgcgcgcagcccgaggtgagcagtgagcggcgagcgggagggcagcgaggcgttcgcgggccccctcctgctgcccgggcccggcccgctcatggcggccatccgcaagaagctggtggtggtgggcgacggcgcgtgtggcaagacgtgcctgctgatcgtgttcagtaaggacgagttccccgaggtgtacgtgcccaccgtcttcgagaactatgtggccgacattgaggtggacggcaagcaggtggagctggcgctgtgggacacggcgggccaggaggactacgaccgcctgcggccgctctcctacccggacaccgacgtcattctcatgtgcttctcggtggacagcccggactcgctggagaacatccccgagaagtgggtccccgaggtgaagcacttctgtcccaatgtgcccatcatcctggtggccaacaaaaaagacctgcgcagcgacgagcatgtccgcacagagctggcccgcatgaagcaggaacccgtgcgcacggatgacggccgcgccatggccgtgcgcatccaagcctacgactacctcgagtgctctgccaagaccaaggaaggcgtgcgcgaggtcttcgagacggccacgcgcgccgcgctgcagaagcgctacggctcccagaacggctgcatcaactgctgcaaggtgctatgagggccgcgcccgtcgcgcctgcccctgccggcacggctccccctcctggaccagtcccccgcgagcccggagaaggggagacccgtgtcccacaaggaccccaccggcctgcctggcatctgtctgctgacgcctctggcttgcgccaggacttggcgtgggcaccgggcgcccccatcccagtgtctgtgtgcgtccagctgtgttgcacaggcctgggctccccactgagtgccaagggtcccctgagcatgcttttctgaagagccgggcctcagagtgtgtggctgtgtgtctgttcgactcccctcgccccattttcaccccacccccgcctctgatccccgggggcgagattggcgcgggagtgtggccgcgccccatcagatgttctcccttcaccagcgggagcttgatatcccttgtctgtaacatagaccccgggtactgcgggaggggagggctgctggggaggatggggggatgttatataaatatagatataattttattttcggagctaagatggtgttatttaagggtggtgatgggtgagcgctctggcccaggctgggccagactcccgcccaagcatgaacaggacttgaccatctttccaacccctggggaagacatttgcaactgacttggggaggacacagcttcagcacagcctctcctgcgggccagcccgctgcgaaccctccaccagctaccggagggaggagggaggatgcgctgtggggttgtttttgccataagcgaactttgtgcctgtcctagaagtgaaaattgttcagtccaagaaactgatgttatttgatttatttaaaggctaaaatttgtttttttattctttgcacaattgtttcattgtttgacacttaatgcactcgtcatttgcatacgacagtagcattctgaccacacttgtacgctgtaacctcatctacttctgatgtttttaaaaaatgacttttaacaaggagagggaaaagaaacccactaaattttgctttgtttccttgaagaatgtggcaacactgttttgtgattttatttgtgcaggtcatgcacacagttttgataaagggcagtaacaagtattggggcctattttttttttttccacaaggcattctctaaagctatgtgaaattttctctgcacctctgtacagagaatacacctgcccctgtatatccttttttcccctcccctccctcccagtggtacttctactaaattgttgtcttgttttttattttttaaataaactgacaaatgacaaaatggtgagcttatgatgtttacataaaagttctataagctgtgtatacagttttttatgtaaaatattaaaagactatgatgatgacatttaaaaaaaaaaaaaaaaaa

SEQ ID NO: 14 - DNA, 875 bp, Homo sapiens
gttatttaagggtggtgatgggtgagcgctctggcccaggctgggccagactcccgcccaagcatgaacaggacttgaccatctttccaacccctggggaagacatttgcaactgacttggggaggacacagcttcagcacagcctctcctgcgggccagcccgctgcgaaccctccaccagctaccggagggaggagggaggatgcgctgtggggttgtttttgccataagcgaactttgtgcctgtcctagaagtgaaaattgttcagtccaagaaactgatgttatttgatttatttaaaggctaaaatttgtttttttattctttgcacaattgtttcattgtttgacacttaatgcactcgtcatttgcatacgacagtagcattctgacccacacttgtacgctggtaacctcatctacttctgatgtttttaaaaaatgacttttaacaaggagagggaaaagaaacccactaaattttgctttgtttccttgaagaatgtggcaacactgttttgtgattttatttgtgcaggtcatgcacacagttttgataaagggcagtaacaagtattggggcctatttttttttttttccacaaggcattctctaaagctatgtgaaattttctctgcacctctgtacagagaatacacctgcccctgtatatccttttttcccctcccctccctcccagtggtacttctactaaattgttgtcttgttttttattttttaaataaactgacaaatgacaaaatggtgagcttatgatgtttacataaaagttctataagctgtgtatacagttttttatgtaaaatattaaaagactatgatgatgacatttaaaaaaaaaaaaaaaaaaa

SEQ ID NO: 15 - DNA, 5032 bp, Homo sapiens
agtgcgacctggccttcttcccctcccccatgcggccggcttccgagtgcgcaagcccagagtcgtccgcgttgtccgcctctgcgcaagcgccgtgctcctcccacggcagttggccatatagaggctgggggtggggggggaggtcaagcgtagcctcttctcctttaccaagatggcggcttgtccctgtttcgccacagttcctaccttatgagctcggttttcttatgcttataagagtggaacagcaaaagctggcaggctgacagaggcggcctcaggacggaccttctggctactgaccgttttgctgtggttttcccggattgtgtgtaggtgtgagatcaaccatgagttccgttgcagttttgacccaagagagttttgctgaacaccgaagtgggctggttccgcaacaaatcaaagttgccactctaaattcagaagaggagagcgaccctccaacctacaaggatgccttccctccacttcctgagaaagctgcttgcctggaaagtgcccaggaacccgctggagcctgggggaacaagatccgacccatcaaggcttctgtcatcactcaggtgttccatgtacccctggaggagagaaaatacaaggatatgaaccagtttggagaaggtgaacaagcaaaaatctgccttgagatcatgcagagaactggtgctcacttggagctgtctttggccaaagaccaaggcctctccatcatggtgtcaggaaagctggatgctgtcatgaaagctcggaaggacattgttgctagactgcagactcaggcctcagcaactgttgccattcccaaagaacaccatcgctttgttattggcaaaaatggagagaaactgcaagacttggagctaaaaactgcaaccaaaatccagatcccacgcccagatgaccccagcaatcagatcaagatcactggcaccaaagagggcatcgagaaagctcgccatgaagtcttactcatctctgccgagcaggacaaacgtgctgtggagaggctagaagtagaaaaggcattccaccccttcatcgctgggccgtataatagactggttggcgagatcatgcaggagacaggcacgcgcatcaacatccccccacccagcgtgaaccggacagagattgtcttcactggagagaaggaacagttggctcaggctgtggctcgcatcaagaagatttatgaggagaagaaaaagaagactacaaccattgcagtggaagtgaagaaatcccaacacaagtatgtcattgggcccaagggcaattcattgcaggagatccttgagagaactggagtttccgttgagatcccaccctcagacagcatctctgagactgtaatacttcgaggcgaacctgaaaagttaggtcaggcgttgactgaagtctatgccaaggccaatagcttcaccgtctcctctgtcgccgccccttcctggcttcaccgtttcatcattggcaagaaagggcagaacctggccaaaatcactcagcagatgccaaaggttcacatcgagttcacagagggcgaagacaagatcaccctggagggccctacagaggatgtcaatgtggcccaggaacagatagaaggcatggtcaaagatttgattaaccggatggactatgtggagatcaacatcgaccacaagttccacaggcacctcattgggaagagcggtgccaacataaacagaatcaaagaccagtacaaggtgtccgtgcgcatccctcctgacagtgagaagagcaatttgatccgcatcgagggggacccacagggcgtgcagcaggccaagcgagagctgctggagcttgcatctcgcatggaaaatgagcgtaccaaggatctaatcattgagcaaagatttcatcgcacaatcattgggcagaagggtgaacggatccgtgaaattcgtgacaaattcccagaggtcatcattaactttccagacccagcacaaaaaagtgacattgtccagctcagaggacctaagaatgaggtggaaaaatgcacaaaatacatgcagaagatggtggcagatctggtggaaaatagctattcaatttctgttccgatcttcaaacagtttcacaagaatatcattgggaaaggaggcgcaaacattaaaaagattcgtgaagaaagcaacaccaaaatcgaccttccagcagagaatagcaattcagagaccattatcatcacaggcaagcgagccaactgcgaagctgcccggagcaggattctgtctattcagaaagacctggccaacatagccgaggtagaggtctccatccctgccaagctgcacaactccctcattggcaccaagggccgtctgatccgctccatcatggaggagtgcggcggggtccacattcactttcccgtggaaggttcaggaagcgacaccgttgttatcaggggcccttcctcggatgtggagaaggccaagaagcagctcctgcatctggcggaggagaagcaaaccaagagtttcactgttgacatccgcgccaagccagaataccacaaattcctcatcggcaaggggggcggcaaaattcgcaaggtgcgcgacagcactggagcacgtgtcatcttccctgcggctgaggacaaggaccaggacctgatcaccatcattggaaaggaggacgccgtccgagaggcacagaaggagctggaggccttgatccaaaacctggataatgtggtggaagactccatgctggtggaccccaagcaccaccgccacttcgtcatccgcagaggccaggtcttgcgggagattgctgaagagtatggcggggtgatggtcagcttcccacgctctggcacacagagcgacaaagtcaccctcaagggcgccaaggactgtgtggaggcagccaagaaacgcattcaggagatcattgaggacctggaagctcaggtgacattagaatgtgctataccccagaaattccatcgatctgtcatgggccccaaaggttccagaatccagcagattactcgggatttcagtgttcaaattaaattcccagacagagaggagaacgcagttcacagtacagagccagttgtccaggagaatggggacgaagctggggaggggagagaggctaaagattgtgaccccggctctccaaggaggtgtgacatcatcatcatctctggccggaaagaaaagtgtgaggctgccaaggaagctctggaggcattggttcctgtcaccattgaagtagaggtgccctttgaccttcaccgttacgttattgggcagaaaggaagtgggatccgcaagatgatggatgagtttgaggtgaacatacatgtcccggcacctgagctgcagtctgacatcatcgccatcacgggcctcgctgcaaatttggaccgggccaaggctggactgctggagcgtgtgaaggagctacaggccgagcaggaggaccgggctttaaggagttttaagctgagtgtcactgtagaccccaaataccatcccaagattatcgggagaaagggggcagtaattacccaaatccggttggagcatgacgtgaacatccagtttcctgataaggacgatgggaaccagccccaggaccaaattaccatcacagggtacgaaaagaacacagaagctgccagggatgctatactgagaattgtgggtgaacttgagcagatggtttctgaggacgtcccgctggaccaccgcgttcacgcccgcatcattggtgcccgcggcaaagccattcgcaaaatcatggacgaattcaaggtggacattcgcttcccacagagcggagccccagaccccaactgcgtcactgtgacggggctcccagagaatgtggaggaagccatcgaccacatcctcaatctggaggaggaatacctagctgacgtggtggacagtgaggcgctgcaggtatacatgaaacccccagcacacgaagaggccaaggcaccttccagaggctttgtggtgcgggacgcaccctggaccgccagcagcagtgagaaggctcctgacatgagcagctctgaggaatttcccagctttggggctcaggtggctcccaagaccctcccttggggccccaaacgataatgatcaaaaagaacagaaccctctccagcctgctgacccaaacccaaccacacaatggtttgtctcaatctgacccagcggctggaccctccgtaaattgttgacgctcttcccccttcccgaggtcccgcagggagcctagcgcctggctgtgtgtgcggccgctcctccaggcctggccgtgcccgctcaggacctgctccactgtttaacactaaaccaaggtcatgagcattcgtgctaagataacagactccagctcctggtccacccggcatgtcagtcagcactctggccttcatcacgagagctccgcagccgtggctaggattccacttcctgtgtcatgacctcaggaaataaacgtccttgactttataaaagccaaacgtttgccctcttcctttcccacctccctcctgccagtttcccttggtccagacagtcctgtttgtggagtgcaatcagcctcctccagctgccagagcgcctcagcacaggtgtcagggtgcaaggaagacctggcaatggacagcaggaggcaggttcctggagctggggggtgacctgagaggcagagggtgacgggttctcaggcagtcctgattttacctgccgtggggtctgaaagcaccaagggtccctgcccctacctccactgccagaccctcagcctgaggtctggtgagtggagcctggaggcaaggtggtaggcaccatctgggtcccctgtggccgtcacagtgtctgctgtgattgagatgcgcacaggttgggggaggtagggccttacgcttgtcctcagtgggggcagtttgccttagatgacagctgggctcttcttcacaccacctgcaggccctccctgcccc

SEQ ID NO: 16 - DNA, 1324 bp, Homo sapiens
cgtggagctgggggtgggcgccgtgtacctaagctggaggctggcgctctccctcagcacaggtgggtcagtggccagcaggcccatctggagtgggagtgggcacttccaccccgcccacaggccatccggctgtgcaggccagcccctaggagcaggtcccgggtgactggcagttttcacggtctagggccgagacgatggcatggggcctagagcatgaggtagagcagaatgcagaccacgccgctggatgccgagagaccctgctctccgagggaggcatctgtgtcatgctgtgagggctgaggacggggccctagtctctggttttctggtcttaacatccttatctgtgtccgccacggaggtgactgagctgctagcgagttgtcctgtcccaggtacttgagttttggaaaagctgactcacgcccatccatctcacagcccttccctggggacagtcgcttccgccttgacacctcactctcagttgaataactcaagcttggtcatcttcagactcgaattcttgagtagacccagacggcttagcccaagtctagttgcagctgcctcggcaagtccccatttgctcaggcagccctgaatgggcctgtttacaggaatggtaaattgggattggaaggaatatagcttccagcttcataggctagggtgaccacggcttaggaaacagggaaagaaagcaaggcccttttcctgcctttcccgggatctgtctactccacctccacgggggaggccagtggggaagggctgtcacctcttccccatctgcatgagttctggaactctgtcctgttggctgcttgcttccagctccccccaatctccatcgcagcgggttcctcctgtcttttctacagtgtcataaaacatcctgcccctaccctctcccaaaggtcaattttaattctcaccaagttttgcacatctctgtatgtcgcttgatgtcttagacgcgagccctttcctaaactgttcagcgctctcttttcctttgggtggttgttgcaagggtgatgacatgactgtccccaggcctgtctccctgaagcgtctgtgctgtcaggacagccctgggcagagatgaggcaggggtgaggcgtgcgtgtgcttttcctccttgttggatgtcttccatatcatctgtttccatagctacaatccatcccttggccttaactttggaatttggagattatatgcaaacatgtgtaaaggctcatgaatatggatgacactggaattttataaattctaaaataaaacccgaaaccagaaaaaaaaaaaaaaa

SEQ ID NO: 17 - DNA, 2100 bp, Homo sapiens
agggcgctccaggcgacagcattgcagacgccattatcctctgtttctctgctgcaccgacctcgacgtcttgcctgtgtcccacttgttcgcggcctataggctactgcagcactggggtgtcagttgttggtccgacccagaacgcttcagttctgctctgcaaggatatataataactgattggtgtgcccgtttaataaaagaatatggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaattaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtatgcagatgttgaaggattctaatgcaagatattttttcttttttatagtgtgaagcagtattctggaaagtttttctaagactagtgaagaactgaaggagtcctgcatctttttttttttatctgcttctgtttaaaaagccaacattcctctgcttcataggtgttctgcatttgaggtgtagtgaaatctttgctgttcaccagatgtaatgttttagttcttacaaacagggttgggggggggaagggcgtgcaaaaactaacattgaaattttgaaacagcagcagagtgagtggattttatttttcgttattgtggtggtttaaaaaattccccccatgtaattattgtgaacaccttgctttgtggtcactgtaacatttggggggtgggccagggaggaaaagtaacaatagtccacatgtccctggcatctgttcagagcagtgtgcagaatgtaatgctcttttgtaagaaacgttttatgatttttaaaataaatttagtgaaccta

SEQ ID NO: 18 - DNA, 2770 bp, Homo sapiens
agggcgctccaggcgacagcattgcagacgccattatcctctgtttctctgctgcaccgacctcgacgtcttgcctgtgtcccacttgttcgcggcctataggctactgcagcactggggtgtcagttgttggtccgacccagaacgcttcagttctgctctgcaaggatatataataactgattggtgtgcccgtttaataaaagaatatggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaattaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtattctggaaagtttttctaagactagtgaagaactgaaggagtcctgcatctttttttttttatctgcttctgtttaaaaagccaacattcctctgcttcataggtgttctgcatttgaggtgtagtgaaatctttgctgttcaccagatgtaatgttttagttcttacaaacagggttgggggggggaagggcgtgcaaaaactaacattgaaattttgaaacagcagcagagtgagtggattttatttttcgttattgtggtggtttaaaaaattccccccatgtaattattgtgaacaccttgctttgtggtcactgtaacatttggggggtgggccagggaggaaaagtaacaatagtccacatgtccctggcatctgttcagagcagtgtgcagaatgtaatgctcttttgtaagaaacgttttatgatttttaaaataaatttagtgaacctatttttggtggtcattttttttttaagacagtcattttaaaatggtggctgaatttcccaacccacccccaaactaaacactaagtttaattttcagctcctctgttggacatataagtgcatctcttgttggacataggcaaaataacttggcaaacttagttctggtgatttcttgatggtttggaagtctattgctgggaagaaattccatcatacatattcatgcttataataagctggggattttttgtttgtttttgcaaatgcttgcccctacttttcaacaattttctatgttagttgtgaagaactaaggtggggagcagtactacaagttgagtaatggtatgagtatataccagaattctgattggcagcaagtttattaatcagaataacacttggttatggaagtgactaatgctgaaaaaattgattatttttattagataatttctcacctatagacttaaactgtcaatttgctctagtgtcttattagttaaactttgtaaaatatatatatacttgtttttccattgtatgcaaattgaaagaaaaagatgtaccatttctctgttgtatgttggattatgtaggaatgtttgtgtacaattcaaaaaaaaaaaagatgaaaaaagttcctgtggatgttttgtgtagtatcttggcatttgtattgatagttaaaattcacttccaaataaataaaacacccatgatgctag

SEQ ID NO: 19 - DNA, 3660 bp, Homo sapiens
gctgtagtcgcggagctgtgtctgttcccaggagtccttcggcggctgttgtgtcgggagcctgatcgcgatggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaatgtgggggtcatcgtggcagccgtccttgtaaccctgattctcctgggaatcttggtttttggcatctggtttgcctatagccgaggccactttgacagaacaaagaaagggacttcgagtaagaaggtgatttacagccagcctagtgcccgaagtgaaggagaattcaaacagacctcgtcattcctggtgtgagcctggtcggctcaccgcctatcatctgcatttgccttactcaggtgctaccggactctggcccctgatgtctgtagtttcacaggatgccttatttgtcttctacaccccacagggccccctacttcttcggatgtgtttttaataatgtcagctatgtgccccatcctccttcatgccctccctccctttcctaccactgctgagtggcctggaacttgtttaaagtgtttattccccatttctttgagggatcaggaaggaatcctgggtatgccattgacttcccttctaagtagacagcaaaaatggcgggggtcgcaggaatctgcactcaactgcccacctggctggcagggatctttgaataggtatcttgagcttggttctgggctctttccttgtgtactgacgaccagggccagctgttctagagcgggaattagaggctagagcggctgaaatggttgtttggtgatgacactggggtccttccatctctggggcccactctcttctgtcttcccatgggaagtgccactgggatccctctgccctgtcctcctgaatacaagctgactgacattgactgtgtctgtggaaaatgggagctcttgttgtggagagcatagtaaattttcagagaacttgaagccaaaaggatttaaaaccgctgctctaaagaaaagaaaactggaggctgggcgcagtggctcacgcctataatcccagaggctgaggcaggcggatcacctgaggtcaggagttcaagatcagcctgaccaacatggagaaaccctactaaaaatacaaagttagccaggcatagtggtgcatgcctgtaatcccagctgctcaggagcctggcaacaagagcaaaactccagctcaaaaaaaaaaagaaagaaaagaaagctggagctggtggcttaggccatcacccttcccttggctggaactactggacagacccttttgagatgtgcctgtggtgctgtggagatgtgtgtagtggtcttagctctttgttgagcttgtgtgtgtgttgtgtagtcttagctgtatgctgaaattgggcgtgtgttggagggcttcttagctctttggtgagattgtatttctatgtgtttgtatcagctgaatgttgctggaaataaaaccttggtttgtcaaggctcttttttgtgggaagtaagtaggggaaaaggtctttgagggttcctaggctcctttgtacaacaggaaaatgcctcaaagccttgcttcccagcaacctggggctggttcccagtgcctggtcctgccccttcctggttcttatctcaaggcagagcttctgaatttcaggccttcattccagagccctcttgtggccaggccttcctttgctggaggaaggtacacagggtgaagctgatgctgtacttgggggatctccttggcctgttccaccaagtgagagaaggtacttactcttgtacctcctgttcagccaggtgcattaacagacctccctacagctgtaggaactactgtcccagagctgaggcaaggggatttctcaggtcatttggagaacaagtgctttagtagtagtttaaagtagtaactgctactgtatttagtggggtggaattcagaagaaatttgaagaccagatcatgggtggtctgcatgtgaatgaacaggaatgagccggacagcctggctgtcattgctttcttcctccccatttggacccttctctgcccttacatttttgtttctccatctaccaccatccaccagtctatttattaacttagcaagaggacaagtaaagggccctcttggcttgattttgcttctttctttctgtggaggatatactaagtgcgactttgccctatcctatttggaaatccctaacagaattgagttttctattaaggatccaaaaagaaaaacaaaatgctaatgaagccatcagtcaagggtcacatgccaataaacaataaattttccagaagaaatgaaatccaactagacaaataaagtagagcttatgaaatggttcagtaaagatgagtttgttgttttttgttttgttttgttttgtttttttaaagacggagtctcgctctgtcacccaggctggagtgcagtggtatgatcttggctcactgtaacctccgcctcccgggttcaagccattctcctgcctcagtctcctgagtagctgggattacgggtgcgtgccaccatgcctggctaatttttgtgtttttagtagagacagggtttcaccatgttggtcgggctggtctcaaactcctgacctcttgatccgcctgccttggcctcccaaagtgatgggattacagatgtgagccaccgtgcctagccaaggatgagatttttaaagtatgtttcagttctgtgtcatggttggaagacagagtaggaaggatatggaaaaggtcatggggaagcagaggtgattcatggctctgtgaatttgaggtgaatggttccttattgtctaggccacttgtgaagaatatgagtcagttattgccagccttggaatttacttctctagcttacaatggaccttttgaactggaaaacaccttgtctgcattcactttaaaatgtcaaaactaatttttataataaatgtttattttcacattgaaaaaaaaaaaaaa

SEQ ID NO: 20 - DNA, 634 bp, Homo sapiens
ccagggccgcactccggagactcgcggttgctacgcgcaccatggctggagcgcccacggtctcgcttcctgaactccgttcactcctagcctccggacgggcccggctcttcgacgtgcgctctcgcgaggaggcggcagctgggaccatcccaggggcgctcaacatcccggtgtccgagttggagagtgctctgcagatggagccagctgccttccaggctttatattctgctgagaagccaaagctggaagatgagcatctcgttttcttctgtcagatgggcaagcggggcctccaggccacgcagctggcccggagtcttggatacactgggtacggggaggtgtggctgctagctgggaggtgatggggactgcctgtcattcctgtcagtctctcacgcttctttgtctccacagggctcgcaactacgctggagcctatagagaatggttggagaaagagagttaggcaggaggcagcttactgattgccaccccctggccccttaatggccaccttaactaagggtgtgaacgggctgacttggtgaattgggcaactccttatagtgttgtgcacacaaaagcatcaaataaagaacatttaatcaaaaaaaaaaaaaaaaaa

SEQ ID NO: 21 - DNA, 2009 bp, Homo sapiens
gctgtagtcgcggagctgtgtctgttcccaggagtccttcggcggctgttgtgtcgggagcctgatcgcgatggggacaaaggcgcaagtcgagaggaaactgttgtgcctcttcatattggcgatcctgttgtgctccctggcattgggcagtgttacagtgcactcttctgaacctgaagtcagaattcctgagaataatcctgtgaagttgtcctgtgcctactcgggcttttcttctccccgtgtggagtggaagtttgaccaaggagacaccaccagactcgtttgctataataacaagatcacagcttcctatgaggaccgggtgaccttcttgccaactggtatcaccttcaagtccgtgacacgggaagacactgggacatacacttgtatggtctctgaggaaggcggcaacagctatggggaggtcaaggtcaagctcatcgtgcttgtgcctccatccaagcctacagttaacatcccctcctctgccaccattgggaaccgggcagtgctgacatgctcagaacaagatggttccccaccttctgaatacacctggttcaaagatgggatagtgatgcctacgaatcccaaaagcacccgtgccttcagcaactcttcctatgtcctgaatcccacaacaggagagctggtctttgatcccctgtcagcctctgatactggagaatacagctgtgaggcacggaatgggtatgggacacccatgacttcaaatgctgtgcgcatggaagctgtggagcggaatgtgggggtcatcgtggcagccgtccttgtaaccctgattctcctgggaatcttggtttttggcatctggtttgcctatagccgaggccactttgacagaacaaagaaagggacttcgagtaagaaggtgatttacagccagcctagtgcccgaagtgaaggagaattcaaacagacctcgtcattcctggtgtgagcctggtcggctcaccgcctatcatctgcatttgccttactcaggtgctaccggactctggcccctgatgtctgtagtttcacaggatgccttatttgtcttctacaccccacagggccccctacttcttcggatgtgtttttaataatgtcagctatgtgccccatcctccttcatgccctccctccctttcctaccactgctgagtggcctggaacttgtttaaagtgtttattccccatttctttgagggatcaggaaggaatcctgggtatgccattgacttcccttctaagtagacagcaaaaatggcgggggtcgcaggaatctgcactcaactgcccacctggctggcagggatctttgaataggtatcttgagcttggttctgggctctttccttgtgtactgacgaccagggccagctgttctagagcgggaattagaggctagagcggctgaaatggttgtttggtgatgacactggggtccttccatctctggggcccactctcttctgtcttcccatgggaagtgccactgggatccctctgccctgtcctcctgaatacaagctgactgacattgactgtgtctgtggaaaatgggagctcttgttgtggagagcatagtaaattttcagagaacttgaagccaaaaggatttaaaaccgcctgccttggcctcccaaagtgatgggattacagatgtgagccaccgtgcctagccaaggatgagatttttaaagtatgtttcagttctgtgtcatggttggaagacagagtaggaaggatatggaaaaggtcatggggaagcagaggtgattcatggctctgtgaatttgaggtgaatggttccttattgtctaggccacttgtgaagaatatgagtcagttattgccagccttggaatttacttctctagcttacaatggaccttttgaactggaaaacaccttgtctgcattcactttaaaatgtcaaaactaatttttataataaatgtttattttcacattgaaaaaaaaaaaaaaaaaaa

SEQ ID NO: 22 - protein, 196 aa, Homo sapiens
MAAIRKKLVVVGDGACGKTCLLIVFSKDEFPEVYVPTVFENYVADIEVDGKQVELALWDTAGQEDYDRLRPLSYPDTDVILMCFSVDSPDSLENIPEKWVPEVKHFCPNVPIILVANKKDLRSDEHVRTELARMKQEPVRTDDGRAMAVRIQAYDYLECSAKTKEGVREVFETATRAALQKRYGSQNGCINCCKVL

SEQ ID NO: 23 - protein, 91 aa, Homo sapiens
MWQHCFVILFVQVMHTVLIKGSNKYWGLFFFFSTRHSLKLCEIFSAPLYREYTCPCISFFPLPSLPVVLLLNCCLVFYFLNKLTNDKMVSL

SEQ ID NO: 24 - protein, 1268 aa, Homo sapiens
MSSVAVLTQESFAEHRSGLVPQQIKVATLNSEEESDPPTYKDAFPPLPEKAACLESAQEPAGAWGNKIRPIKASVITQVFHVPLEERKYKDMNQFGEGEQAKICLEIMQRTGAHLELSLAKDQGLSIMVSGKLDAVMKARKDIVARLQTQASATVAIPKEHHRFVIGKNGEKLQDLELKTATKIQIPRPDDPSNQIKITGTKEGIEKARHEVLLISAEQDKRAVERLEVEKAFHPFIAGPYNRLVGEIMQETGTRINIPPPSVNRTEIVFTGEKEQLAQAVARIKKIYEEKKKKTTTIAVEVKKSQHKYVIGPKGNSLQEILERTGVSVEIPPSDSISETVILRGEPEKLGQALTEVYAKANSFTVSSVAAPSWLHRFIIGKKGQNLAKITQQMPKVHIEFTEGEDKITLEGPTEDVNVAQEQIEGMVKDLINRMDYVEINIDHKFHRHLIGKSGANINRIKDQYKVSVRIPPDSEKSNLIRIEGDPQGVQQAKRELLELASRMENERTKDLIIEQRFHRTIIGQKGERIREIRDKFPEVIINFPDPAQKSDIVQLRGPKNEVEKCTKYMQKMVADLVENSYSISVPIFKQFHKNIIGKGGANIKKIREESNTKIDLPAENSNSETIIITGKRANCEAARSRILSIQKDLANIAEVEVSIPAKLHNSLIGTKGRLIRSIMEECGGVHIHFPVEGSGSDTVVIRGPSSDVEKAKKQLLHLAEEKQTKSFTVDIRAKPEYHKFLIGKGGGKIRKVRDSTGARVIFPAAEDKDQDLITIIGKEDAVREAQKELEALIQNLDNVVEDSMLVDPKHHRHFVIRRGQVLREIAEEYGGVMVSFPRSGTQSDKVTLKGAKDCVEAAKKRIQEIIEDLEAQVTLECAIPQKFHRSVMGPKGSRIQQITRDFSVQIKFPDREENAVHSTEPVVQENGDEAGEGREAKDCDPGSPRRCDIIIISGRKEKCEAAKEALEALVPVTIEVEVPFDLHRYVIGQKGSGIRKMMDEFEVNIHVPAPELQSDIIAITGLAANLDRAKAGLLERVKELQAEQEDRALRSFKLSVTVDPKYHPKIIGRKGAVITQIRLEHDVNIQFPDKDDGNQPQDQITITGYEKNTEAARDAILRIVGELEQMVSEDVPLDHRVHARIIGARGKAIRKIMDEFKVDIRFPQSGAPDPNCVTVTGLPENVEEAIDHILNLEEEYLADVVDSEALQVYMKPPAHEEAKAPSRGFVVRDAPWTASSSEKAPDMSSSEEFPSFGAQVAPKTLPWGPKR

SEQ ID NO: 25 - protein, 464 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 26 - protein, 463 aa, Homo sapiens
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 27 - protein, 299 aa, Homo sapiens
MGTKAQVERKLLCLFILAILLCSLALGSVTVHSSEPEVRIPENNPVKLSCAYSGFSSPRVEWKFDQGDTTRLVCYNNKITASYEDRVTFLPTGITFKSVTREDTGTYTCMVSEEGGNSYGEVKVKLIVLVPPSKPTVNIPSSATIGNRAVLTCSEQDGSPPSEYTWFKDGIVMPTNPKSTRAFSNSSYVLNPTTGELVFDPLSASDTGEYSCEARNGYGTPMTSNAVRMEAVERNVGVIVAAVLVTLILLGILVFGIWFAYSRGHFDRTKKGTSSKKVIYSQPSARSEGEFKQTSSFLV

SEQ ID NO: 28 - protein, 122 aa, Homo sapiens
RAALRRLAVATRTMAGAPTVSLPELRSLLASGRARLFDVRSREEAAAGTIPGALNIPVSELESALQMEPAAFQALYSAEKPKLEDEHLVFFCQMGKRGLQATQLARSLGYTGYGEVWLLAGR

(19) United States
(12) Patent Application Publication
(10) Pub. No.: US 2007/0059726 A1
(43) Pub. Date: Mar. 15, 2007

(54) Differential transcript expression

(76) Inventor: Xiaohong Cao, Southborough, MA (US)

Correspondence Address: GENZYME CORPORATION;LEGAL DEPARTMENT, 15 PLEASANT ST CONNECTOR, FRAMINGHAM, MA, 01701-9322, US

(21) Appl. No.: 11/405,999

(22) Filed: Apr. 18, 2006

Related U.S. Application Data
(60) Provisional application No. 60/672,080, filed on Apr. 18, 2005.

(51) Int. Cl. C12Q 1/68; G01N 33/574; C07H 21/04

(52) U.S. Cl. 435/6; 435/7.23; 536/24.3

(57) ABSTRACT

Multiple transcripts from the same gene maybe differentially regulated. Finding such differential regulation under distinct physiological conditions implicates the gene in the generation or response to the physiological condition. Transcripts have been identified from five genes which appear to be differentially regulated in lung cancer and normal cells. We have also identified a set of transcripts from a gene which are differentially regulated in squamous cell lung cancer from lung adenocarcinoma. The technique employed to identify these differentially regulated transcripts can be applied to other physiological conditions and samples to identify other differentially regulated transcripts.

### CROSS-REFERENCE TO RELATED APPLICATIONS

This application claims priority under 35 U.S.C. § 119(e) to U.S. Provisional Application No. 60/672,080, filed Apr. 18, 2005, the contents of which are herein incorporated by reference.

### TECHNICAL FIELD OF THE INVENTION

This invention is related to the area of cancer diagnostics. In particular, it relates to independent regulation of distinct transcripts of particular genes.

### BACKGROUND OF THE INVENTION

Alternative splicing has been recognized as a widespread event in mammalian gene expression. Estimates of alternative splicing frequency have ranged from 40-60% of human genes with an average of three variants per gene. Different splice variants of a particular gene are often specific to different stages of development and particular tissues. Disruption of pre-mRNA splicing has also been shown to cause various genetic diseases.

Lung cancer is the uncontrolled growth of abnormal cells in one or both of the lungs. While normal lung tissue cells reproduce and develop into healthy lung tissue, these abnormal cells reproduce rapidly and never grow into normal lung tissue. Lumps of cancer cells (tumors) then form and disrupt the lung, making it difficult to function properly.

The two main types of lung cancer are non-small cell (80% of all cases) and small cell (20% of all cases). The names refer to the kinds of cells that make up the tumor rather than the size of the tumor. Non-small cell lung cancer is classified into three subtypes: adenocarcinomas found in the mucus glands; squamous or epidermoid carcinoma located in the bronchial tubes; and large cell carcinoma found near the surface.

Lung cancer almost always begins in one lung and, if left untreated, can spread to lymph nodes or other tissues in the chest (including the other lung). Lung cancer can also metastasize throughout the body, to the bones, brain, liver, or other organs.

Early detection of lung cancer is critical to improving chances of survival. The five-year survival rate for those whose lung cancer is found when it is localized (before it has spread to other organs) is nearly 50%. Only 15% of lung cancer cases are found at the localized early stage. When lung cancer is detected in an early-stage and surgery is possible, the five-year survival rates can reach 85%.

A number of different tests are used to detect and diagnose lung cancer, including sophisticated imaging scans that provide more accurate and sensitive results than conventional X-rays. The information from these tests enables the physician to determine the type and stage of the cancer and the best way to treat it. Currently employed tests include: physical examination, chest examination, chest X-ray, computer tomography (CT) scan, positron emission tomography (PET) scan, Magnetic Resonance Imaging (MRI), sputum cytology, bronchoscopy, biopsy.

There is a continuing need in the art for additional diagnostic techniques for lung cancers so that more lung cancers can be detected earlier and survival rates can be improved.

### SUMMARY OF THE INVENTION

According to one embodiment of the invention a method is provided for diagnosing cancer in a lung tissue sample. One compares (a) expression level of a splice variant transcript of a gene in a test lung tissue sample of a patient to (b) expression level of the splice variant transcript in a normal lung tissue sample. The transcript is selected from the group consisting of: transcript 3 of F11R (SEQ ID NO: 20), transcript 2 of RAS Homolog Gene Family Member B, (SEQ ID NO: 14), and transcript 1 of each of High Density Lipoprotein (SEQ ID NO: 15), Hypothetical Protein FLJ21918, HNRPK (SEQ ID NO: 17), and F11 receptor (SEQ ID NO: 19). One identifies the lung tissue sample as cancerous if the expression level is higher in the test sample than in the normal sample.

According to another embodiment of the invention a second method is provided for diagnosing cancer in a lung tissue sample. One compares (a) expression level of a splice variant transcript of a gene in a test lung tissue sample of a patient to (b) expression level of the splice variant transcript in a normal lung tissue sample. The splice variant transcript is selected from the group consisting of: transcript 1 of Ras Homolog Gene Family, Member B (SEQ ID NO: 13), and transcript 2 of each of High Density Lipoprotein (SEQ ID NO: 16), Hypothetical Protein FLJ21918, Heterogeneous Nuclear Ribonucleoprotein K (SEQ ID NO: 18), and F11 receptor (SEQ ID NO: 21). One identifies the lung tissue sample as cancerous if the expression level is lower in the test sample than in the normal sample.

Another method provided by the invention is for diagnosing cancer in a lung tissue sample. One compares (a) expression level of a splice variant transcript of a gene in a test lung tissue sample of a patient to (b) expression level of the splice variant transcript in a normal lung tissue sample. The splice variant transcript comprises a tag sequence located at a position 3′ of the 3′-most site for an NlaIII restriction endonuclease in a cDNA reverse transcribed from the splice variant transcript. The tag sequence is selected from the group consisting of: SEQ ID NO: 1, 3, 5, 7, 9, 10, and 12. One identifies the lung tissue sample as cancerous if the expression level is higher in the test sample than in the normal sample.

Another aspect of the invention is a fourth method for diagnosing cancer in a lung tissue sample. One compares (a) expression level of a splice variant transcript of a gene in a test lung tissue sample of a patient to (b) expression level of the splice variant transcript in a normal lung tissue sample. The splice variant transcript comprises a tag sequence located at a position 3′ of the 3′-most site for an NlaIII restriction endonuclease in a cDNA reverse transcribed from the splice variant transcript, wherein the tag sequence is selected from the group consisting of SEQ ID NO: 2, 4, 6, 8, and 11. One identifies the lung tissue sample as cancerous if the expression level is lower in the test sample than in the normal sample.

Yet another aspect of the invention is a fifth method for diagnosing cancer in a lung tissue sample. One compares (a) expression level of a first splice variant transcript of a gene in a test lung tissue sample of a patient to (b) expression level of a second splice variant transcript of the gene in the test lung tissue sample. The first splice variant transcript is selected from the group consisting of: transcript 3 of F11R (SEQ ID NO: 20), transcript 2 of RAS Homolog Gene Family Member B, (SEQ ID NO: 14), and transcript 1 of each of High Density Lipoprotein (SEQ ID NO: 15), Hypothetical Protein FLJ21918, HNRPK (SEQ ID NO: 17), and F11 receptor (SEQ ID NO: 19). The second splice variant transcript is selected from the group consisting of transcript 1 of Ras Homolog Gene Family, Member B (SEQ ID NO: 13), and transcript 2 of each of High Density Lipoprotein (SEQ ID NO: 16), Hypothetical Protein FLJ21918, Heterogeneous Nuclear Ribonucleoprotein K (SEQ ID NO: 18), and F11 receptor (SEQ ID NO: 21). One identifies the lung tissue sample as cancerous if the expression level of the first splice variant transcript is higher than expression of the second splice variant transcript in the test sample. One identifies the lung tissue sample as normal if the expression level of the first splice variant transcript is lower than expression of the second splice variant transcript in the test sample.

Still another aspect of the invention is a sixth method for diagnosing cancer in a lung tissue sample. One compares (a) expression level of a first splice variant transcript of a gene in a test lung tissue sample of a patient to (b) expression level of a second splice variant transcript of the gene in the test lung tissue sample. The first and second splice variant transcripts comprise a tag sequence located at a position 3′ of the 3′-most site for an NlaIII restriction endonuclease in a cDNA reverse transcribed from the transcript. The tag sequence for the first splice variant transcript is selected from the group consisting of: SEQ ID NO: 1, 3, 5, 7, 9, 10, and 12. The tag sequence for the second splice variant transcript is selected from the group consisting of SEQ ID NO: 2, 4, 6, 8, and 11. One identifies the lung tissue sample as cancerous if the expression level of the first splice variant sequence is higher in the test sample than the expression level of the second splice variant sequence. One identifies the lung tissue sample as normal if the expression level of the first splice variant transcript is lower than expression of the second splice variant transcript in the test sample.

A probe is provided by the present invention. The probe comprises a polynucleotide consisting essentially of any one of SEQ ID NO: 1-12 or its complement. The probe also comprises a label or a moiety for binding a label with a high affinity.

A seventh embodiment of the invention provides an isolated and purified polynucleotide comprising a cDNA of a Heterogeneous Nuclear Ribonucleoprotein K transcript. The cDNA comprises SEQ ID NO: 7 located at a position 3′ of the 3′-most site for an NlaIII restriction endonuclease on the cDNA.

Yet another method is provided by the present invention. This method is for determining sample-specific expression of splice variants of a gene. One obtains SAGE tag library expression data for a matched set of tissues comprising a first and a second tissue. One applies a correction algorithm to the data which eliminates spurious tags in the expression data which do not correspond to actual transcripts in the matched set of tissues. One compares expression level of at least two splice variant transcripts from a single gene in the first tissue to expression level of the at least two splice variant transcripts in the second tissue. One identifies splice variants as having sample-specific expression if a first splice variant transcript of the gene is expressed higher in the first tissue than in the second tissue and if a second splice variant transcript of the gene is expressed higher in the second tissue than in the first tissue.

The present invention further provides a method of distinguishing lung squamous cell carcinoma from lung adenocarcinoma. One compares the level of transcript 1 to transcript 2 of F11R in a test sample. Transcript 1 comprises SEQ ID NO: 10 and transcript 2 comprises SEQ ID NO: 11, each of said sequences located at a position 3′ of the 3′-most site for a NlaIII restriction endonuclease in a cDNA reverse transcribed from the respective transcript. One identifies the test sample as squamous cell carcinoma if the ratio of the levels of transcript 1 to transcript 2 is greater than 1.5:1, and identifying the test sample as adenocarcinoma if the ratio of the levels of transcript 1 to transcript 2 is less than 1:1.

Still another method provide by the present invention is for diagnosing cancer in a lung tissue sample. One compares (a) expression level of a protein product of a splice variant transcript of a gene in a test lung tissue sample of a patient to (b) expression level of a protein product of the splice variant transcript in a normal lung tissue sample. The protein product of the transcript is selected from the group consisting of: transcript 3 of F11R, transcript 2 of RAS Homolog Gene Family, Member B (SEQ ID NO: 23), and transcript 1 of each of High 3Density Lipoprotein (SEQ ID NO: 24), Hypothetical Protein FLJ21918, HNRPK (SEQ ID NO: 25), and F11 receptor (SEQ ID NO: 27). The lung tissue sample is identified as cancerous if the expression level is higher in the test sample than in the normal sample.

A further method is provided for diagnosing cancer in a lung tissue sample. One compares (a) expression level of a protein product of a splice variant transcript of a gene in a test lung tissue sample of a patient to (b) expression level of the protein product of the splice variant transcript in a normal lung tissue sample. The protein product of the transcript is selected from the group consisting of: transcript 1 of Ras Homolog Gene Family, Member B (SEQ ID NO: 22), and transcript 2 of each of Hypothetical Protein FLJ21918, Heterogeneous Nuclear Ribonucleoprotein K (SEQ ID NO: 26), and F11 receptor (SEQ ID NO: 28). One identifies the lung tissue sample as cancerous if the expression level is lower in the test sample than in the normal sample.

Yet another method for diagnosing cancer in a lung tissue sample is provided by the invention. One compares (a) expression level of a protein product of a first splice variant transcript of a gene in a test lung tissue sample of a patient to (b) expression level of a protein product of a second splice variant transcript of the gene in the test lung tissue sample. The protein product of the first splice variant transcript is selected from the group consisting of: transcript 3 of F11R, transcript 2 of RAS Homolog Gene Family, Member B (SEQ ID NO: 23), and transcript 1 of each of High Density Lipoprotein (SEQ ID NO: 24), Hypothetical Protein FLJ21918, HNRPK (SEQ ID NO: 25), and F11 receptor (SEQ ID NO: 27). The protein product of the second splice variant transcript is selected from the group consisting of transcript 1 of Ras Homolog Gene Family, Member B (SEQ ID NO: 22), and transcript 2 of each of Hypothetical Protein FLJ21918, Heterogeneous Nuclear Ribonucleoprotein K (SEQ ID NO: 26), and F11 receptor (SEQ ID NO: 28). One identifies the lung tissue sample as cancerous if the expression level of the protein product of the first splice variant transcript is higher than expression of the protein product of the second splice variant transcript in the test sample. One identifies the lung tissue sample as normal if the expression level of the protein product of the first splice variant transcript is lower than expression of the protein product of the second splice variant transcript in the test sample.

Still another embodiment of the invention is a method of distinguishing a lung squamous cell carcinoma from a lung adenocarcinoma. One compares the level of a protein product of transcript 1 of F11R to the level of a protein product of transcript 2 of F11R in a test sample. The protein product of the transcript 1 comprises SEQ ID NO: 27, and the protein product of the transcript 2 comprises SEQ ID NO: 28. One identifies the test sample as squamous cell carcinoma if the ratio of protein product of transcript 1 to protein product of transcript 2 is greater than 1.5:1. One identifies the test sample as adenocarcinoma if the ratio of protein product of transcript 1 to protein product of transcript 2 is less than 1:1.

These and other embodiments which will be apparent to those of skill in the art upon reading the specification provide the art with reagents and methods for detection and diagnosis of lung cancers.

### BRIEF DESCRIPTION OF THE DRAWINGS

FIG. 1 shows a summary of the tags (SEQ ID NO: 1-12) and ratios in different tumor libraries.

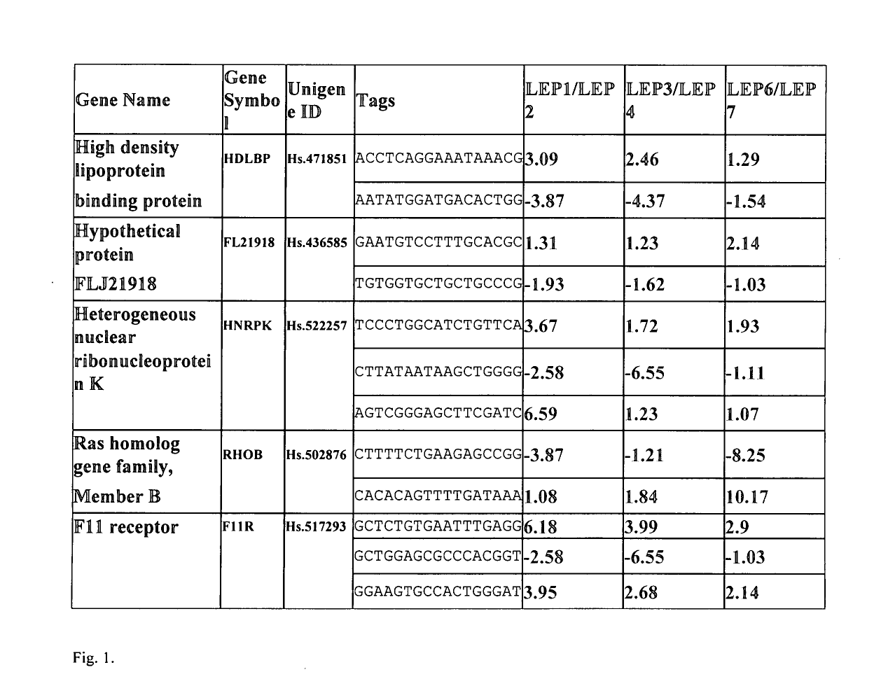

FIG. 2 shows tag ratios and absolute tag counts for HNRPK.

FIG. 3 shows tag ratios and absolute tag counts for F11R.

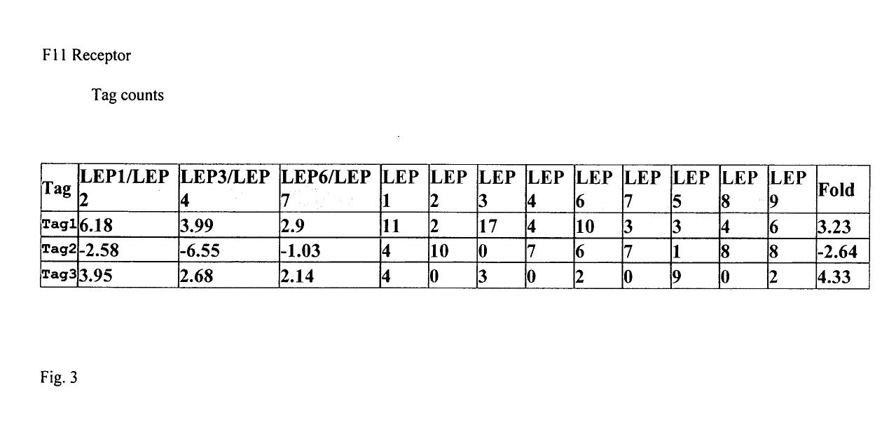

FIG. 4 shows tag ratios and absolute tag counts for HDLBP.

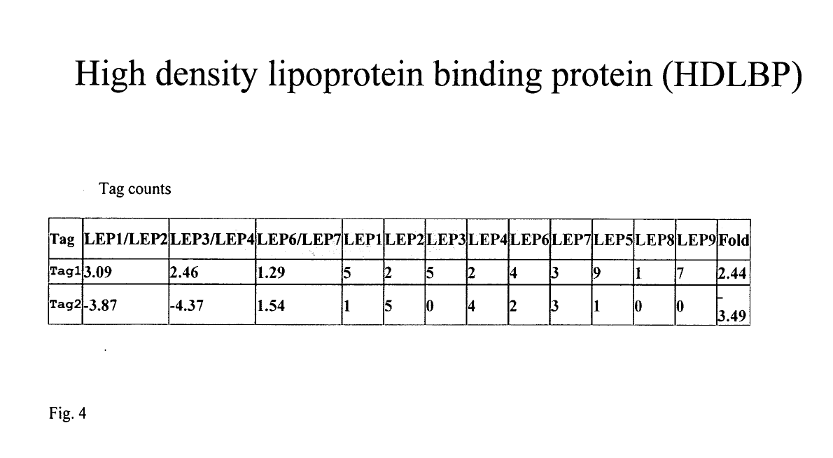

FIG. 5 shows tag ratios and absolute tag counts for RHOB

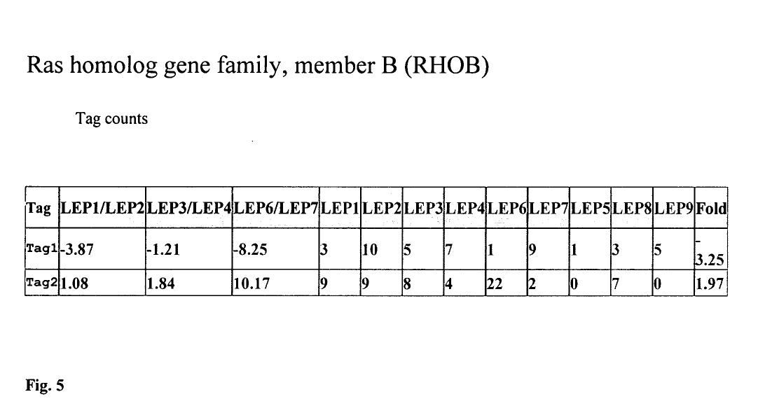

FIG. 6 shows the correlation of tags to transcripts to protein sequences.

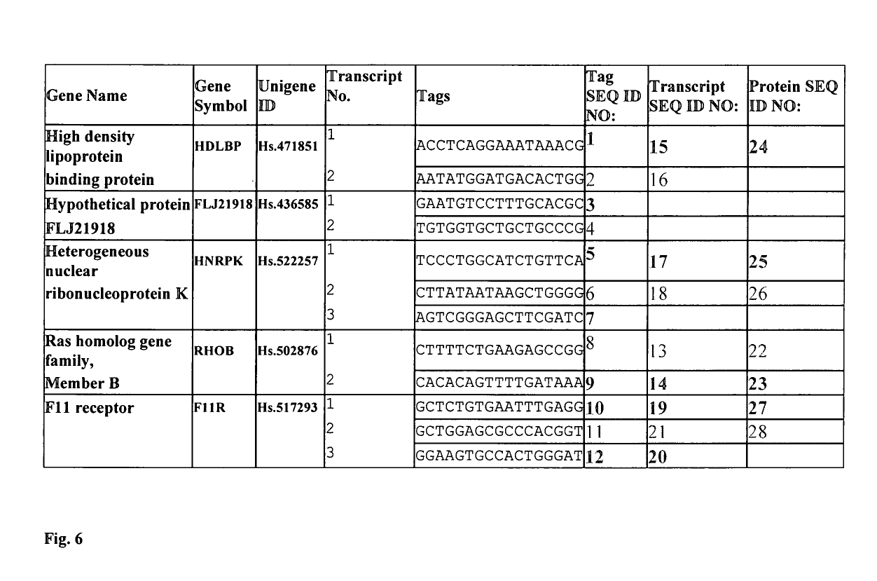

FIG. 7 shows a summary of the samples used to make the tag libraries.

The sequence listing filed herewith forms part of the disclosure of the present application.

### DETAILED DESCRIPTION OF THE INVENTION

We have examined alternative splice variant expression during tumor formation via Serial Analysis of Gene Expression (SAGE). Matched tumor and normal epithelial cell samples were isolated from lung cancer patients and used to generate SAGE tag libraries. The differential expression pattern of each gene transcript was studied, and the expression levels of transcripts corresponding to the same gene were compared. We have observed genes showing elevated expression of one splice variant in cancer samples but elevated expression of a different splice variant in normal samples. The expression patterns of transcripts in adenocarcinoma samples do not always follow those in the squamous cell samples. These results indicate that differential transcript expression can be used to distinguish types of tumor tissue and to distinguish tumor from normal. Moreover, the methods used to uncover these patterns can be used to discover more patterns useful for other types of cancer detection.

Serial Analysis of Gene Expression (SAGE) (Velculescu et al., 1995) is a protocol for systematic, high-throughput generation of short expressed sequence tags (ESTs) from a cell sample, producing a global profile of gene expression. Briefly, SAGE generates short mRNA sequence tags from a specific position in transcripts. The tag position is defined by the location of the 3′-most anchoring enzyme restriction site. The most commonly used enzyme for this purpose is NlaIII. cDNA fragments from cleavage with an anchoring enzyme are further processed with the tagging enzyme, a Type IIS restriction endonuclease, typically BsmFI. Following amplification, cloning and sequencing, the end result of a SAGE experiment is a set of vector inserts from which ditag and, ultimately, tag sequences are extracted and counted. Each SAGE tag is prefixed by the anchoring enzyme restriction site and corresponds to the 10-11 bp extension of the 3′-most site in the cognate transcript. In theory, tags of this length are sufficiently specific to map the transcriptome. In fact, most human SAGE tags map uniquely to the Uni-Gene clusters (Lash et al., 2000). Given such a bi-directional map, expression levels of the transcripts are inferred from observations of their SAGE tags. Recently, the SAGE protocol was enhanced with a new tagging enzyme. This enzyme, MmeI, cuts 21-22 bases downstream of the anchoring enzyme restriction site (Saha et al., 2002). The new protocol, Long SAGE, enhances the specificity of SAGE to transcriptome mapping and allows direct mapping of Long SAGE tags to the genome.

The SAGE protocol is subject to sequence errors introduced by the polymerase chain reaction (PCR) and sequencing steps. Sub-optimal fidelity in these procedures can introduce artifact tag sequences. Such errors occur infrequently for any individual transcript and have little effect on the quantification of differential expression of moderately expressed genes. Their consequence is greater on the measurement of rare transcripts and the identification of novel genes. In addition, accumulation of such spurious tags introduces noise into the overall profile of transcripts in a sample and obfuscates the characterization of transcriptome size. An algorithm can be used to eliminate spurious tags from the dataset. One such algorithm which can be used is embodied in a software program called SAGEScreen. See Akmaev and Wang, Bioinformatics, 20: 1254-1263 (2004), the disclosure of which is expressly incorporated herein. Preferably the algorithm will remove at least 10%, at least 20%, at least 30%, at least 40%, at least 50%, at least 60%, of the unique tags in a tag library.

While the multiple transcripts from a single gene that we identified may represent splice variants resulting from differential processing of precursor mRNAs (pre-mRNAs), in some cases they may also be the result of using different polyadenylylation (polyA) signals. We refer to different transcripts from the same gene as splice variants herein, without regard to how they were actually formed.

Expression levels of different transcripts are compared in order to determine whether the source lung tissue is cancerous or normal, and in some cases whether the tissue is one type of lung cancer or another. Expression levels can be compared in a clinical sample using any technique known in the art. Messenger RNA levels can be examined or protein levels can be examined.

Clinical samples can be from biopsies, surgically removed organs, autopsy tissue, sputum, etc. Any source of lung cells can be used. The samples can be prepared for the technique that will be used for examining expression. For example, if in situ hybridization is to be performed, then appropriate histological samples will be prepared and/or used. Cellular extracts may be appropriate for use in immunological assays for protein products. The skilled artisan will be able to readily prepare the clinical tissue for the analysis technique to be performed.

Determining whether expression is higher or lower in one sample than another will vary based on the technique employed. Statistically significant changes are typically used to make a determination of higher or lower expression. Background values will vary based on the techniques employed. The differences between two samples will be at least 20%, at least 30%, at least 40%, at least 50%, at least 75%, at least 100%, or at least 200% higher or lower. Some comparisons, such as between transcripts 1 and 2 of F11R, employ a ratio cut off. If the ratio is greater than 1.25:1, greater than 1.5:1, or greater than 2:1, then the sample is identified as being squamous cell carcinoma. If the ratio is less than 1.25:1, less than 1.1:1, or less than 1:1, then the sample is identified as adenocarcinoma.

Control normal samples can be obtained from the same patient as the test tissue, or they can be obtained from other normal individuals or from panels of other normal individuals.

Techniques which can be used for comparing expression levels are many. Any technique for accomplishing expression assays can be used. These include without limitation hybridization to probes on arrays, quantitative PCR, RT-PCR, SAGE analysis, in situ hybridization, immunohistochemistry, ELISA assays, and Western blots. If transcript expression is being assayed, the SAGE tags disclosed herein can be used as probes or as parts of probes. Such probes may contain additional sequences that do not interfere with the hybridization of the probe. Typically additional sequences will be on the termini of the tags. Additional sequences may be additional complementary nucleotides to the desired transcript or they may be added for other functions, such as linkers, or restriction sites, or binding sites, etc. Probes typically have an element that serves as a means of detection. The element can be a radioisotopic label, a fluorescent moiety, a bioluminescent moiety, a chemiluminescent moiety, a binding partner, such as biotin, streptavidin, or avidin. Such binding partners are typically high affinity binding partners which bind with an avidity similar to or stronger than an antigen to its specific antibody. Those of skill in the art readily understand and can make the choice of a means for detection. Probes that are described as “consisting essentially of” a certain stated sequence typically contain at most an additional <10, <7, <5, <3, or 1 nucleotide on either or both termini. Such additional nucleotides will preferably not interfere with the binding of the probe to its corresponding mRNA or cDNA.

One new splice variant has been identified for Heterogeneous Nuclear Ribonucleoprotein K (HNRPK) in the course of this study. SEQ ID NO: 7 identifies the new transcript. The cDNA for this transcript can be isolated and purified as is known in the art. For example, a probe comprising the sequence shown in SEQ ID NO: 7 can be used to hybridize to and purify the transcript or its cDNA. The cDNA can be used in vectors, for example, in order to express the encoded protein.

Other condition-specific splice variant expression sets can be identified as described herein. Tissues that can be compared in addition to cancer versus normal, and one type of cancer versus a second type of cancer, include tissues at different developmental stages, tissues of different developmental lineages, tissues that have been differentially treated, for example with or without a drug, candidate drug, toxin, or other biologically active agent.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

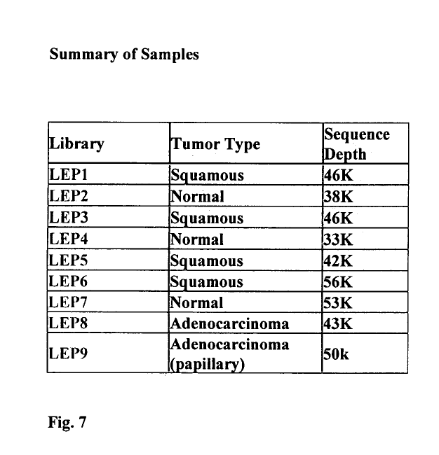

We compared expression in three sets of paired samples (tumor to normal) from individuals with squamous cell lung carcinoma. In addition, we studied three additional unpaired lung tumor samples (one from squamous cell cancer and two from adenocarcinomas). See FIG. 7. We used the SAGE technique to obtain tags. Mapping was used to identify different tags as representing the same gene as follows:

UniGene Map Generation Steps

1. Download current build of UniGene

2. Pull out the mRNA and 3′ labeled EST sequences from each cluster

3. Extract tags from the pulled sequences

4. Generate tag summary and cluster summary file

Cluster Summary

| | | | | Num of | # mRNA | # EST | |
|---|---|---|---|---|---|---|---|
| Unigene | Tag | Total mRNA | Total EST | Tags | with the tag | with the tag | Flag |
| Hs.278741 | CGCCTCTCCAGCCTTCA | 3 | 1 | 2 | 3 | 0 | Yes |
| Hs.278741 | TCATCCTGATCAAAGAC | 3 | 1 | 0 | 2 | 1 | |

Flag is “yes” if ([# mRNA with the tag]+[# EST with the tag]) >=([Total mRNA]+[Total EST])/[Num of Tags]

Tag Summary

| | | | | | | | | |
|---|---|---|---|---|---|---|---|---|
| | Total | Total | Total | Num of | | mRNA from | EST from | |
| Tag | Tag Num | mRNA | EST | Clusters | Unigene | the cluster | the cluster | Flag |

| | | | | | | | | |
|---|---|---|---|---|---|---|---|---|
| AAATAAAGCACCCACA | 1 | 0 | 1 | 1 | Hs.300697 | 0 | 1 | yes |

Flag is “yes” if ([# mRNA from the cluster]+[#EST from the cluster]) >=([Total mRNA]+[Total EST])/[Num of Clusters]

5. Pull out the flagged UniGene clusters and tags to build the gene2tag_map and tag2gene_map.

Five genes were identified that showed transcript specific regulation associated with cancer and that fit our strict criteria for consistency among cancer patients. While the multiple transcripts from a single gene that we identified may represent splice variants resulting from differential processing of precursor mRNAs (pre-mRNAs), in some cases they may also be the result of using different polyadenylylation (polyA) signals. We refer to different transcripts from the same gene as splice variants herein, without regard to how they were actually formed.

### EXAMPLE 2

### Heterogeneous Nuclear Ribonucleoprotein K

Heterogeneous nuclear ribonucleoprotein K (HNRPK) belongs to the subfamily of ubiquitously expressed heterogeneous nuclear ribonucleoproteins (hnRNPs). These are RNA binding proteins, which complex with heterogeneous nuclear RNA (hnRNA). The hnRNPs associate with pre-mRNAs in the nucleus. They appear to influence pre-mRNA processing and other aspects of mRNA metabolism and transport. HnRNPs are thought to have a role during cell cycle progression. While multiple alternatively spliced transcript variants have been described for this gene, only three variants have been fully described.

HNRPK Tag 1 corresponds to transcript variant 1 and tag 2 corresponds to transcript variant 2. The expression pattern of HNRPK tag 1 and tag 2 are the same in squamous and adenocarcinoma samples.

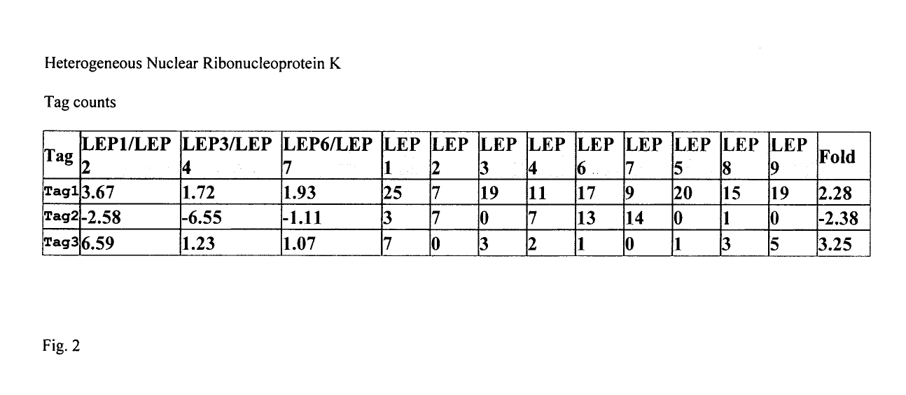

Results are shown in FIG. 2.

### EXAMPLE 3

### F11 Receptor

F11 Receptor belongs to the immunoglobulin superfamily. It is an important regulator of tight junction assembly in epithelia. F11R acts as: (1) a receptor for reovirus, (2) a ligand for the integrin LFA1, involved in leukocyte transmigration, and (3) a platelet receptor. Five transcript variants encoding two different isoforms have been found for this gene.

Our observations support the existence of additional splice variants in addition to the ones identified in current database. Usage of alternative poly A sites at 3′ UTR seems to be the mechanism that controls the expression. The expression pattern of F11R tags differ in squamous and adenocarcinoma samples, thus these tags can be used to distinguish lung squamous cell from adenocarcinoma.

I/we claim:

1. A method for diagnosing cancer in a lung tissue sample, comprising:
comparing (a) expression level of a splice variant transcript of a gene in a test lung tissue sample of a patient to (b) expression level of the splice variant transcript in a normal lung tissue sample, wherein the transcript is selected from the group consisting of: transcript 3 of F11R (SEQ ID NO: 20), transcript 2 of RAS Homolog Gene Family Member B, (SEQ ID NO: 14), and transcript 1 of each of High Density Lipoprotein (SEQ ID NO: 15), Hypothetical Protein FLJ21918, Heterogeneous Nuclear Ribonucleoprotein K (SEQ ID NO: 17), and F11 receptor (SEQ ID NO: 19);
identifying the lung tissue sample as cancerous if the expression level is higher in the test sample than in the normal sample.

2. The method of claim 1 wherein the normal lung tissue sample is from the patient.

3. A method for diagnosing cancer in a lung tissue sample, comprising:
comparing (a) expression level of a splice variant transcript of a gene in a test lung tissue sample of a patient to (b) expression level of the splice variant transcript in a normal lung tissue sample, wherein the transcript is selected from the group consisting of: transcript 1 of Ras Homolog Gene Family, Member B (SEQ ID NO: 13), and transcript 2 of each of High Density Lipoprotein (SEQ ID NO: 16), Hypothetical Protein FLJ21918, Heterogeneous Nuclear Ribonucleoprotein K (SEQ ID NO: 18), and F11 receptor (SEQ ID NO: 21);
identifying the lung tissue sample as cancerous if the expression level is lower in the test sample than in the normal sample.

4. The method of claim 3 wherein the normal lung tissue sample is from the patient.

5. A method for diagnosing cancer in a lung tissue sample, comprising:
comparing (a) expression level of a splice variant transcript of a gene in a test lung tissue sample of a patient to (b) expression level of the splice variant transcript in a normal lung tissue sample, wherein the splice variant transcript comprises a tag sequence located at a position 3′ of the 3′-most site for a NlaIII restriction endonuclease in a cDNA reverse transcribed from the splice variant transcript, wherein the tag sequence is selected from the group consisting of: SEQ ID NO: 1, 3, 5, 7, 9, 10, and 12;
identifying the lung tissue sample as cancerous if the expression level is higher in the test sample than in the normal sample.

6. The method of claim 5 wherein the normal lung tissue sample is from the patient.

7. A method for diagnosing cancer in a lung tissue sample, comprising:
comparing (a) expression level of a splice variant transcript of a gene in a test lung tissue sample of a patient to (b) expression level of the splice variant transcript in a normal lung tissue sample, wherein the splice variant transcript comprises a tag sequence located at a position 3′ of the 3′-most site for a NlaIII restriction endonuclease in a cDNA reverse transcribed from the splice variant transcript, wherein the tag sequence is selected from the group consisting of SEQ ID NO: 2, 4, 6, 8, and 11;
identifying the lung tissue sample as cancerous if the expression level is lower in the test sample than in the normal sample.

8. The method of claim 7 wherein the normal lung tissue sample is from the patient.

9. The method of claim 1 wherein expression level is determined using a probe comprising a sequence as shown in any one of the group consisting of SEQ ID NO: 1, 3, 5, 7, 9, 10, and 12.

10. The method of claim 3 wherein expression level is determined using a probe comprising a sequence as shown in any one of the group consisting of SEQ ID NO: 2, 4, 6, 8, and 11.

11. The method of claim 5 wherein expression level is determined using a probe comprising a sequence as shown in any one of the group consisting of SEQ ID NO: 1, 3, 5, 7, 9, 10, and 12.

12. The method of claim 7 wherein expression level is determined using a probe comprising a sequence as shown in any one of the group consisting of SEQ ID NO: 2, 4, 6, 8, and 11.

13. A method for diagnosing cancer in a lung tissue sample, comprising:
comparing (a) expression level of a first splice variant transcript of a gene in a test lung tissue sample of a patient to (b) expression level of a second splice variant transcript of the gene in the test lung tissue sample, wherein the first splice variant transcript is selected from the group consisting of: transcript 3 of F11R (SEQ ID NO: 20), transcript 2 of RAS Homolog Gene Family Member B, (SEQ ID NO: 14), and transcript 1 of each of High Density Lipoprotein (SEQ ID NO: 15), Hypothetical Protein FLJ21918, Heterogeneous Nuclear Ribonucleoprotein K (SEQ ID NO: 17), and F11 receptor (SEQ ID NO: 19), and wherein the second splice variant transcript is selected from the group consisting of transcript 1 of Ras Homolog Gene Family, Member B (SEQ ID NO: 13), and transcript 2 of each of High Density Lipoprotein (SEQ ID NO: 16), Hypothetical Protein FLJ21918, Heterogeneous Nuclear Ribonucleoprotein K (SEQ ID NO: 18), and F11 receptor (SEQ ID NO: 21);
identifying the lung tissue sample as cancerous if the expression level of the first splice variant transcript is higher than expression of the second splice variant transcript in the test sample, and identifying the lung tissue sample as normal if the expression level of the first splice variant transcript is lower than expression of the second splice variant transcript in the test sample.

14. A method for diagnosing cancer in a lung tissue sample, comprising:
comparing (a) expression level of a first splice variant transcript of a gene in a test lung tissue sample of a patient to (b) expression level of a second splice variant transcript of the gene in the test lung tissue sample, wherein the first and second splice variant transcripts comprise a tag sequence located at a position 3′ of the 3′-most site for a NlaIII restriction endonuclease in a cDNA reverse transcribed from the transcript, wherein the tag sequence for the first splice variant transcript is selected from the group consisting of: SEQ ID NO: 1, 3, 5, 7, 9, 10, and 12 and wherein the tag sequence for the second splice variant transcript is selected from the group consisting of SEQ ID NO: 2, 4, 6, 8, and 11;
identifying the lung tissue sample as cancerous if the expression level of the first splice variant sequence is higher in the test sample than the expression level of the second splice variant sequence, and identifying the lung tissue sample as normal if the expression level of the first splice variant transcript is lower than expression of the second splice variant transcript in the test sample.

15. The method of any of claims 1, 3, 5, 7 wherein the expression levels of at least two of said splice variant transcripts are compared.

16. The method of any of claims 1, 3, 5, 7 wherein the expression levels of at least three of said splice variant transcripts are compared.

17. The method of any of claims 1, 3, 5, 7 wherein the expression levels of at least four of said splice variant transcripts are compared.

18. A probe comprising:
a polynucleotide consisting essentially of any one of SEQ ID NO: 1-12 or its complement; and
a label or a moiety for binding a label with a high affinity.

19. The probe of claim 18 wherein the label is a radioisotopic label.

20. The probe of claim 18 wherein the label is a fluorescent moiety.

21. The probe of claim 18 wherein the label is a bioluminescent moiety.

22. The probe of claim 18 wherein the moiety is selected from the group consisting of biotin, streptavidin, and avidin.

23. An isolated and purified polynucleotide comprising a cDNA of a Heterogeneous Nuclear Ribonucleoprotein K transcript, said cDNA comprising SEQ ID NO: 7 located at a position 3′ of the 3′-most site for an NlaIII restriction endonuclease on the cDNA.

24. A method of determining sample-specific expression of splice variants of a gene, comprising:
obtaining SAGE tag library expression data for a matched set of tissues comprising a first and a second tissue;
applying a correction algorithm to the data which eliminates spurious tags in the expression data which do not correspond to actual transcripts in the matched set of tissues;
comparing expression level of at least two splice variant transcripts from a single gene in the first tissue to expression level of the at least two splice variant transcripts in the second tissue;
identifying splice variants as having sample-specific expression if a first splice variant transcript of the gene is expressed higher in the first tissue than in the second tissue and a second splice variant transcript of the gene is expressed higher in the second tissue than in the first tissue.

25. The method of claim 24 wherein the first tissue is a pathological tissue and the second tissue is a normal tissue of the same tissue type.

26. The method of claim 24 wherein the first tissue is a neoplastic tissue and the second tissue is a normal tissue of the same tissue type.

27. The method of claim 24 wherein the first tissue and the second tissue comprise cells of the same lineage at different developmental stages.

28. The method of claim 24 wherein the first tissue and the second tissue comprise cells of different lineages at the same developmental stage.

29. The method of claim 24 wherein the first tissue and the second tissue comprise cells of a single type which have been differentially treated.

30. The method of claim 24 further comprising the steps of:
mapping tags in the SAGE tag library to a database of mRNA and/or expressed sequence tag (EST) sequences; and
identifying two tags which map to the same gene, whereby the two tags are determined to represent splice variant transcripts of a single gene.

31. A method of distinguishing lung squamous cell carcinoma from lung adenocarcinoma, comprising:
comparing the level of transcript 1 to transcript 2 of F11R in a test sample, wherein transcript 1 comprises SEQ ID NO: 10 and transcript 2 comprises SEQ ID NO: 11, each of said sequences located at a position 3′ of the 3′-most site for a NlaIII restriction endonuclease in a cDNA reverse transcribed from the respective transcript;
identifying the test sample as squamous cell carcinoma if the ratio of the levels of transcript 1 to transcript 2 is greater than 1.5:1, and identifying the test sample as adenocarcinoma if the ratio of the levels of transcript 1 to transcript 2 is less than 1:1.

32. A method for diagnosing cancer in a lung tissue sample, comprising:
comparing (a) expression level of a protein product of a splice variant transcript of a gene in a test lung tissue sample of a patient to (b) expression level of a protein product of the splice variant transcript in a normal lung tissue sample, wherein the protein product of the transcript is selected from the group consisting of: transcript 3 of F11R, transcript 2 of RAS Homolog Gene Family, Member B (SEQ ID NO: 23), and transcript 1 of each of High Density Lipoprotein (SEQ ID NO: 24), Hypothetical Protein FLJ21918 (SEQ ID NO:), Heterogeneous Nuclear Ribonucleoprotein K (SEQ ID NO: 25), and F11 receptor (SEQ ID NO: 27);
identifying the lung tissue sample as cancerous if the expression level is higher in the test sample than in the normal sample.

33. The method of claim 32 wherein the normal lung tissue sample is from the patient.

34. A method for diagnosing cancer in a lung tissue sample, comprising:
comparing (a) expression level of a protein product of a splice variant transcript of a gene in a test lung tissue sample of a patient to (b) expression level of the protein product of the splice variant transcript in a normal lung tissue sample, wherein the protein product of the transcript is selected from the group consisting of: transcript 1 of Ras Homolog Gene Family, Member B (SEQ ID NO: 22), and transcript 2 of each of Hypothetical Protein FLJ21918, Heterogeneous Nuclear Ribonucleoprotein K (SEQ ID NO: 26), and F11 receptor (SEQ ID NO: 28);
identifying the lung tissue sample as cancerous if the expression level is lower in the test sample than in the normal sample.

35. The method of claim 34 wherein the normal lung tissue sample is from the patient.

36. The method of claim 32 or 34 wherein expression level is determined using an antibody which binds to an epitope that is present in one protein product encoded by a first splice variant but not present in a second protein encoded by a second splice variant.

37. A method for diagnosing cancer in a lung tissue sample, comprising:
comparing (a) expression level of a protein product of a first splice variant transcript of a gene in a test lung tissue sample of a patient to (b) expression level of a protein product of a second splice variant transcript of the gene in the test lung tissue sample, wherein the protein product of the first splice variant transcript is selected from the group consisting of: transcript 3 of F11R, transcript 2 of RAS Homolog Gene Family, Member B (SEQ ID NO: 23), and transcript 1 of each of High Density Lipoprotein (SEQ ID NO: 24), Hypothetical Protein FLJ21918, Heterogeneous Nuclear Ribonucleoprotein K (SEQ ID NO: 25), and F11 receptor (SEQ ID NO: 27), and wherein the protein product of the second splice variant transcript is selected from the group consisting of transcript 1 of Ras Homolog Gene Family, Member B (SEQ ID NO: 22), and transcript 2 of each of Hypothetical Protein FLJ21918, Heterogeneous Nuclear Ribonucleoprotein K (SEQ ID NO: 26), and F11 receptor (SEQ ID NO: 28);
identifying the lung tissue sample as cancerous if the expression level of the protein product of the first splice variant transcript is higher than expression of the protein product of the second splice variant transcript in the test sample, and
identifying the lung tissue sample as normal if the expression level of the protein product of the first splice variant transcript is lower than expression of the protein product of the second splice variant transcript in the test sample.

38. A method of distinguishing a lung squamous cell carcinoma from a lung adenocarcinoma, comprising:
comparing the level of a protein product of transcript 1 of F11R to the level of a protein product of transcript 2 of F11R in a test sample, wherein the protein product of the transcript 1 comprises SEQ ID NO: 27 and the protein product of the transcript 2 comprises SEQ ID NO: 28;
identifying the test sample as squamous cell carcinoma if the ratio of protein product of transcript 1 to protein product of transcript 2 is greater than 1.5:1, and identifying the test sample as adenocarcinoma if the ratio of protein product of transcript 1 to protein product of transcript 2 is less than 1:1.
